(19) United States
(12) Patent Application Publication
(10) Pub. No.: US 2013/0018481 A1
(43) Pub. Date: Jan. 17, 2013

(54) COATED STENTS AND PROCESS FOR COATING WITH PROTEIN

(71) Applicants: Thomas Subkowski, Ladenburg (DE); Uwe Weickert, Bad Rappenau (DE)

(72) Inventors: Thomas Subkowski, Ladenburg (DE); Uwe Weickert, Bad Rappenau (DE)

(73) Assignee: BASF SE, Ludwigshafen (DE)

(21) Appl. No.: 13/637,720

(22) PCT Filed: Mar. 30, 2011
(86) PCT No.: PCT/EP2011/054887

§ 371 Date: Sep. 27, 2012

Related U.S. Application Data
(60) Provisional application No. 61/319,274, filed on Mar. 31, 2010.

(30) Foreign Application Priority Data
Mar. 31, 2010 (EP) 10158592.5

(51) Int. Cl. A61K 38/02; B05D 7/00; A61P 7/02; A61F 2/04

(52) U.S. Cl. 623/23.7; 427/2.25; 514/1.1

(57) ABSTRACT

A process for coating of implantable structures, such as stents, by using a composition which comprises at least one hydrophobin derivative (H), water and further components leads to long time usable implants.

The invention relates to coated medical devices, such as implantable structures, e.g. stents, and to new processes for coating implantable structures, e.g. stents, and other medical devices with a protein. The invention also relates to processes for coating medical devices, such as stents, anastomotic devices and perivascular wraps with a composition containing a particular protein, hydrophobin, either alone or hydrophobin in combination with other components, such as heparin.

A wide range of implantable structures, e.g. stents, is known, in particular biliary stents. Plastic and metal stents are used for various purposes, but high quality stents with low cost of production, permanent function and easy handling of use and exchange are not available.

The present invention relates to medical devices, such as implantable structures (I), which are coated with hydrophobin and which in a particular embodiment can be used e.g. for the local delivery of a drug or drug combinations, e.g. for the prevention and treatment of vascular diseases. The present invention also relates to medical devices, including stents, anastomotic devices, perivascular wraps, sutures and staples being coated with hydrophobin. These medical devices can be used to treat and prevent diseases and minimize or substantially eliminate a biological organism's reaction to the introduction of the medical device to the organism. In addition, the coated devices can be utilized to promote healing and endothelialization.

The present invention also relates to coatings for medical devices, in particular implantable structures such as stents. The present invention also covers coatings for controlling the elution rates of drugs, agents and/or compounds from implantable medical devices. The present invention also relates to drug delivery systems for the regional delivery of drugs, such as for treating vascular disease. The present invention also relates to coated medical devices, such as implantable structures, having a hydrophobin and a drug affixed thereto for treating diseases.

Hydrophobins and the derivatives are small proteins of from about 100 to 150 amino acids, which occur in filamentous fungi such as Schizophyllum commune. They generally have 8 cysteine units (Cys) in the molecule. Hydrophobins are among the most surface-active proteins of fungal origin. Hydrophobins contain diverse amino acid sequences, which are sharing a characteristic pattern of eight Cys residues in their primary sequence by forming four disulfide bridges. The disulfide bridges formed by Cys residues are known to account for the controlled assembly at hydrophilic-hydrophobic interfaces preventing spontaneous self-assembly in solution. These proteins are found to be important for aerial growth (e.g., aerial hyphae, spores and fruiting bodies such as mushrooms) and for the attachment of fungi to solid supports. Hydrophobins are remarkably stable and can withstand temperatures near the boiling point of water. Hydrophobins can be isolated from natural sources but they can also be obtained by means of recombinant methods, as disclosed for example in WO 2006/082 251 or WO 2006/131 564. The prior art has already proposed the use of several hydrophobins for various applications. WO 1996/41882 proposes the use of hydrophobins as emulsifiers and thickeners for hydrophilizing hydrophobic surfaces. It also has been proposed to use hydrophobins as a demulsifier, see WO 2006/103251, as an evaporation retardant, see WO 2006/128877 or as soiling inhibitor, see WO 2006/103215. A method of deposition of hydrophobin derivatives on different surfaces, such as plastic polymeric surfaces, glass, metallic surfaces, naturally surfaces like leather, cotton and paper, from aqueous solution is described in WO 2006/082253 and EP-A 1 252 516.

The present invention relates to the coating of medical devices with a hydrophobin. A particular aspect of the invention relates to a new process for coating of an implantable structure (I) comprising the steps of treating the surface of the implantable structure (I) with a composition which comprises at least one hydrophobin derivative (H).

The invention also relates to a process for coating of an implantable structure (I), characterized in that the implantable structure (I) is a stent and that the surface is treated with a composition which comprises at least one hydrophobin derivative (H) and at least one further component (F).

The invention also relates to a process for coating of an implantable structure (I), wherein the composition comprises at least one hydrophobin derivative (H) and water, and potentially further components (F), whereby the amount of the hydrophobin derivative (H), based on the overall composition, is from 0.0001 to 20 percent, often from 0.001 to 10 percent by weight. On the surface of the implantable structure (I), the amount of the hydrophobin derivative is often in the range of 0.1 to 10 mg/m².

Further components can be e.g. polymeric additives, solvents, buffer, pharmaceutically active substances and/or auxiliaries. The term auxiliaries encompasses a pharmaceutically acceptable, physiologically inactive ingredient such as a binder, a filler and a coatingforming compound. Further examples of optional auxiliaries excipients are anti-adhesives, preservatives, glidants, lubricants and sorbents. Suitable substances are known in the art.

Often, the composition is a water-based composition. The invention also relates to a process for coating of an implantable structure (I) wherein the implantable structure (I) is a stent which is treated with a composition which comprises at least one hydrophobin derivative (H), water and potentially further components (F), whereby the amount of the hydrophobin derivative (H), based on the overall composition, is from 0.001 to 10 percent by weight.

The invention also relates to a process for coating of an implantable structure (I) wherein a composition is applied to the surface of the implantable structure (I) which comprises the hydrophobin derivative (H), water and at least one further pharmaceutically active compound (D). The process can encompass further steps such as cleaning and watering steps.

The invention also relates to a process for coating of an implantable structure (I) wherein a composition is applied to the surface of the implantable structure (I) which comprises the hydrophobin derivative (H), water and as further pharmaceutically active compound (D) one or several compounds from the group comprising heparin, antibiotics (such as ampicillin or sulbactam or levofloxacin) and cytostatic compounds (such as alkylantia, anti-metabolites, mitosis-inhibitors or hormones). The combination of hydrophobin and heparin and its derivatives (such as enoxaparin) is of particular interest. Further examples are compositions comprising a hydrophobin and a cumarin-derivative, such as Warfarin, Phenprocoumon or Ethylbiscoumacetat. The further pharmaceutically active compound (D) can also be chemically linked to the hydrophobin.

The invention also relates to a process for coating of an implantable structure (I) wherein the hydrophobin derivative (H) used is a fusion hydrophobin or a derivative thereof. The invention also relates to a process for coating of an implantable structure (I) wherein the composition comprising at least one hydrophobin derivative (H) is applied to the surface of the implantable structure (I) at a temperature from 4° C. to 95° C., in particular 20° C. to 90° C., for a time period of 0.01 hour to 48 hours, in particular 0.1 to 20 hours, often from 1 to 10 hours.

A further aspect of this invention is the coated implantable structure (I) with a surface at least partially treated with a hydrophobin derivative (H).

The invention also relates to a coated implantable structure (I) which is at least partially surface coated with a hydrophobin derivative (H) by a process as described above.

The invention also relates to a coated implantable structure (I) wherein the implantable structure (I) is a stent, in particular a biliary stent.

An additional object of the invention is the providing of a composition for the coating of implantable structures (I), wherein the composition comprises based on the total composition 0.0001 to 20 percent by weight of hydrophobin (H) and 99.999 to 80 percent by weight of further components (F). As further component, the solvent water is often used.

The invention also relates to a composition for the coating of implantable structures (I) wherein the composition comprises at least 0.001 to 10 percent by weight of at least one hydrophobin derivative (H), at least 50 percent by weight of water and potentially further components (F). A further aspect is the use of a hydrophobin derivative (H) for the coating of an implantable structure (I), in particular of a stent.

Several types of medical devices such as implantable structures (I) are often coated before use. As one example of implantable structures, biliary stents are used to treat obstructions that occur in the bile ducts. Bile is a substance that helps to digest fats and is produced by the liver, secreted through the bile ducts and stored in the gallbladder. It is released into the small intestine after a fat-containing meal has been eaten. There are a number of conditions, malignant or benign, that can cause strictures of the bile duct. Pancreatic cancer is a common malignant cause, cancers of the gallbladder, bile duct, liver and large intestine are further examples. Non-cancerous causes of bile duct stricture include injury to the bile ducts during surgery for gallbladder removal, pancreatitis (inflammation of the pancreas), primary sclerosing cholangitis (an inflammation of the bile ducts), gallstones, radiation therapy and blunt trauma to the abdomen.

A biliary stent often is a thin, tube-like structure which can be surface-coated and which is used to support a narrowed part of the bile duct and prevent the reformation of the stricture. Stents can be made e.g. of plastic or metal. The two most common methods used to place a biliary stent are endoscopic retrograde cholangio-pancreatography (ERCP) and percutaneous transhepatic cholangiography (PTC). For both methods it can be of advantage to use coated devices. The ERCP is an imaging technique used to diagnose diseases of the pancreas, liver, gallbladder, and bile ducts that also has the advantage of being used as a therapeutic device. The endoscope is a thin, lighted, hollow tube, attached to a viewing screen and can be inserted into a patient's mouth, down the esophagus, through the stomach, and into the upper part of the small intestine, until it reaches the spot where the bile ducts empty. At this point a small tube called a cannula is inserted through the endoscope and used to inject a contrast dye into the ducts. A series of x rays are then taken as the dye moves through the ducts. If the x rays show that a biliary stricture exists, a coated stent may be placed into a duct to relieve the obstruction. In order to do this, special instruments are inserted into the endoscope and a sphincterotomy is performed to provide access to the bile ducts. In some cases, the biliary stricture may first be dilated using a thin, flexible tube called catheter, followed by a balloon-type device that is inflated. The coated stent is then inserted into the bile duct. The other method for applying coated stents, percutaneous transhepatic cholangiography or PTC, is similar to ERCP in that the test is used to diagnose and treat obstructions affecting the flow of bile from the liver to the gastrointestinal tract. A thin needle is used to inject a contrast dye through the skin and into the liver or gallbladder. X rays pictures are taken while the dye moves through the bile ducts. If a biliary stricture becomes evident, a coated stent may then be placed. A hollow needle is introduced into the bile duct, and a thin guide wire inserted into the needle.

The wire is guided to the area of obstruction and the coated stent is advanced over the wire and placed in the obstructed duct.

Stents can also be used in other medical fields.

Many individuals suffer from circulatory disease caused by a progressive blockage of the blood vessels that perfuse the heart and other major organs. More severe blockage of blood vessels often leads to hypertension, ischemic injury, stroke, or myocardial infarction. Atherosclerotic lesions are a major cause of ischemic heart disease. The percutaneous transluminal coronary angioplasty is a medical procedure whose purpose is to increase blood flow through an artery. Percutaneous transluminal coronary angioplasty is the predominant treatment for coronary vessel stenosis. A limitation associated with percutaneous transluminal coronary angioplasty is the abrupt closure of the vessel, which may occur immediately after the procedure and restenosis, which occurs gradually following the procedure.

Numerous agents have been tested with stents as anti-proliferative actions in restenosis and have shown some activity in experimental animal models. Some agents which have been shown to successfully reduce the extent of intimal hyperplasia in animal models include heparin and heparin fragments, taxol, angiotensin converting enzyme inhibitors, angiopeptin, cyclosporine A, terbinafine, interferon-gamma, rapamycin, steroids, antisense oligionucleotides and gene vectors. Coated stents can also be used in reducing restenosis.

These coated stents are often balloon-expandable slotted metal tubes (e.g. stainless steel), which when expanded within the lumen of an angioplastied coronary artery provide structural support through rigid scaffolding to the arterial wall. This support is helpful in maintaining vessel lumen patency. The increased angiographic success of these coated stents after percutaneous transluminal coronary angioplasty can be shown. Additionally, the coating of stents with hydrophobin and eventually further compounds, such as heparin and its derivatives, appears to have the added benefit of producing a reduction in subacute thrombosis after stent implantation. The coating of stents with hydrophobin and heparin (and eventually other compounds) can have clinical usefulness.

The use of hydrophobin coated stents was also found to be a way of local drug delivery. The manner in which the particular drug (or drug combination) is affixed to the local delivery device, e.g. the stent, plays a role in the efficacy of this type of treatment.

One typical way according to the invention is to chemically fixate the drug molecule to the protein hydrophobin which is coated onto the surface of the device. The process and materials utilized to affix the drug (or drug combination) to the stent should not interfere with the operations of the drug. In addition, the processes and materials utilized should be biocompatible and maintain the drug or drug combination on the local device through delivery and over a long period of time. There is a need for specially protein coated stents which can be used as local delivery devices for the prevention and treatment of diseases, such as vascular injury.

A variety of methods for stent coating and compositions have been proposed. The coatings may be capable themselves of reducing the stimulus the stent provides to the injured part (e.g. lumen wall), thus reducing the tendency towards thrombosis or restenosis. Alternately, the coating may deliver a pharmaceutical drug. The mechanism for delivery of the drug is e.g. through diffusion of the agent through a bulk polymer or through pores that are created in the polymer structure, or by erosion of a biodegradable coating. The drug compound can also be coated on the surface of the stent by using the protein hydrophobin.

Both bio-absorbable and biostable compositions have been reported as coating materials for stents. Polymeric coatings can encapsulate a pharmaceutical drug. Other ways of binding such an agent to the surface are known, e.g. heparin-coated stents. These coatings are applied to the stent in a number of ways, including dip, spray, or spin coating processes. One class of biostable polymeric materials that has been reported as coatings for stents is polyfluoro homopolymers. Polytetrafluoroethylene (PTFE) homopolymers have been used as implants for many years. These homopolymers are not soluble in any solvent at reasonable temperatures and therefore are difficult to coat onto small medical devices while maintaining important features of the devices (e.g. slots in stents). Stents with coatings made from polyvinylidenefluoride homopolymers and containing pharmaceutical/therapeutic agents or drugs for release have been suggested. However, they are difficult to apply as high quality films onto surfaces without subjecting them to relatively high temperatures.

It now was found to be advantageous to develop coatings for implantable structures containing hydrophobin or derivatives thereof.

These coatings can be applied to the surface of the medical device by using compositions containing hydrophobin. These coatings can be applied to implantable medical devices that may include, but do not require, the use of pharmaceutical agents or drugs. These coated devices possess physical and mechanical properties effective for use. It can be advantageous to use the coated medical devices in combination with drugs which treat disease and minimize or substantially eliminate a living organisms' reaction to the implantation of the medical device. In certain circumstances, it is advantageous to develop hydrophobin-coated medical devices in combination with drugs, such as heparin, which promote wound healing and endothelialization of the medical device.

In the context of the present invention, the term “hydrophobins” or “hydrophobin derivates” can be understood to mean in particular polypeptides of the general structural formula (I)

Xₙ—C¹—X₁₋₅₀—C²—X₀₋₅—C³—X₁₋₁₀₀—C⁴—X₁₋₁₀₀—C⁵—X₁₋₅₀—C⁶—X₁₋₅—C⁷—X₁₋₅₀—C⁸—Xₘ (I)

where X may be any of the 20 naturally occurring amino acids (Phe, Leu, Ser, Tyr, Cys, Trp, Pro, His, Gln, Arg, Ile, Met, Thr, Asn, Lys, Val, Ala, Asp, Glu, Gly). In the formula, the X radicals may be the same or different in each case. The indices beside X are each the number of amino acids in the particular part-sequence X, C is cysteine, alanine, serine, glycine, methionine or threonine, where at least four of the residues designated with C are cysteine, and the indices n and m are each independently natural numbers between 0 and 500, preferably between 15 and 300. The polypeptides of the formula (I) are also characterized by the property that, at room temperature, after coating a glass surface, they bring about an increase in the contact angle of a water droplet of at least 20°, preferably at least 25° and more preferably 30°, compared in each case with the contact angle of an equally large water droplet with the uncoated glass surface.

The amino acids designated with C¹ to C⁸ are preferably cysteines. However, they may also be replaced by other amino acids with similar space-filling, preferably by alanine, serine, threonine, methionine or glycine.

However, at least four, preferably at least 5, more preferably at least 6 and in particular at least 7 of positions C¹ to C⁸ should consist of cysteines. In the inventive proteins, cysteines may either be present in reduced form or form disulfide bridges with one another. Particular preference is given to the intramolecular formation of C—C bridges, especially that with at least one intramolecular disulfide bridge, preferably 2, more preferably 3 and most preferably 4 intramolecular disulfide bridges. In the case of the above-described exchange of cysteines for amino acids with similar space-filling, such C positions are advantageously exchanged in pairs which can form intramolecular disulfide bridges with one another. If cysteines, serines, alanines, glycines, methionines or threonines are also used in the positions designated with X, the numbering of the individual C positions in the general formulae can change correspondingly.

Preference is given to using hydrophobins of the general formula (II)

Xₙ—C¹—X₃₋₂₅—C²—X₀₋₂—C³—X₅₋₅₀—C⁴—X₂₋₃₅—C⁵—X₂₋₁₅—C⁶—X₀₋₂—C⁷—X₃₋₃₅—C⁸—Xₘ (II)

to perform the present invention, where X, C and the indices beside X and C are each as defined above, the indices n and m are each numbers between 0 and 350, preferably from 15 to 300, and the proteins additionally feature the above-illustrated change in contact angle, and, furthermore, at least 6 of the residues designated with C are cysteine. More preferably, all C residues are cysteine.

Particular preference is given to using hydrophobins of the general formula (III)

Xₙ—C¹—X₅₋₉—C²—C³—X₁₁₋₃₉—C⁴—X₂₋₂₃—C⁵—X₅₋₉—C⁶—C⁷—X₆₋₁₈—C⁸—Xₘ (III)

where X, C and the indices beside X are each as defined above, the indices n and m are each numbers between 0 and 200, and the proteins additionally feature the above-illustrated change in contact angle, and at least 6 of the residues designated with C are cysteine. More preferably, all C residues are cysteine. The Xₙ and Xₘ residues may be peptide sequences which naturally are also joined to a hydrophobin. However, one residue or both residues may also be peptide sequences which are naturally not joined to a hydrophobin.

This is also understood to mean those Xₙ and/or Xₘ residues in which a peptide sequence which occurs naturally in a hydrophobin is lengthened by a peptide sequence which does not occur naturally in a hydrophobin.

If Xₙ and/or Xₘ are peptide sequences which are not naturally bonded to hydrophobins, such sequences are generally at least 20, preferably at least 35 amino acids in length. They may, for example, be sequences of from 20 to 500, preferably from 30 to 400 and more preferably from 35 to 100 amino acids. Such a residue which is not joined naturally to a hydrophobin will also be referred to hereinafter as a fusion partner. This is intended to express that the proteins may consist of at least one hydrophobin moiety and a fusion partner moiety which do not occur together in this form in nature. Fusion hydrophobins composed of fusion partner and hydrophobin moiety are described, for example in WO 2006/082251, WO 2006/082253 and WO 2006/131564.

The fusion partner moiety may be selected from a multitude of proteins. It is possible for only one single fusion partner to be bonded to the hydrophobin moiety, or it is also possible for a plurality of fusion partners to be joined to one hydrophobin moiety, for example on the amino terminus (Xₙ) and on the carboxyl terminus (Xₘ) of the hydrophobin moiety. However, it is also possible, for example, for two fusion partners to be joined to one position (Xₙ or Xₘ) of the inventive protein. Particularly suitable fusion partners are proteins which naturally occur in microorganisms, especially in Escherischia coli or Bacillus subtilis. Examples of such fusion partners are the sequences yaad (SEQ ID NO: 16 in WO 2006/082251), yaae (SEQ ID NO: 18 in WO 2006/082251), ubiquitin and thioredoxin.

Also very suitable are fragments or derivatives of these sequences which comprise only some, for example from 70 to 99%, preferentially from 5 to 50% and more preferably from 10 to 40% of the sequences mentioned, or in which individual amino acids or nucleotides have been changed compared to the sequence mentioned, in which case the percentages are each based on the number of amino acids. Instead of the complete fusion partner, it may be advantageous to use a truncated residue.

In particular the truncated residue can comprise at least 20, preferably at least 35 amino acids. In a further preferred embodiment, the fusion hydrophobin, as well as the fusion partner mentioned as one of the Xₙ or Xₙ, groups or as a terminal constituent of such a group, also have a so-called affinity domain (affinity tag/affinity tail). In a manner known in principle, this comprises anchor groups which can interact with particular complementary groups and can serve for easier workup and purification of the proteins. Examples of such affinity domains comprise (His)ₖ, (Arg)ₖ, (Asp)ₖ, (Phe)ₖ or (Cys)ₖ groups, where k is generally a natural number from 1 to 10. It may preferably be a (His)ₖ group, where k is from 4 to 6. In this case, the Xₙ and/or Xₘ group may consist exclusively of such an affinity domain, or else an Xₙ or Xₘ radical which is or is not naturally bonded to a hydrophobin is extended by a terminal affinity domain.

The hydrophobins used in accordance with the invention may also be modified in their polypeptide sequence, for example by glycosylation, acetylation or else by chemical crosslinking, for example with glutaraldehyde. The hydrophobins can also be crosslinked with a polysaccharide, such as heparin.

It was found to be advantageous to develop coated devices that are not adversely affecting the coating by implanting the medical device. In addition, such hydrophobin-coated devices provide the physician with a means for easily and accurately positioning the medical device in the target area. It was also found advantageous to develop hydrophobin coatings for medical devices that allow for the precise control of the elution rate of a drug (such as heparin) from the medical devices.

Hydrophobin coated devices that provide for the release of one or more agents that act through different molecular mechanisms affecting cell proliferation, are also subject of the invention.

For testing purposes, commercially available biliary plastic stents were coated either with hydrophobin alone or with hydrophobin and antibiotics and/or heparin. After an incubation period of 28 days in human bile, it was investigated, if the clogging material on the surface of the coated stents was reduced. It was found that the coating of the plastic stents with hydrophobin led to a significant reduction of the adhering material on the surface of the stents.

Coupling of ampicillin/sulbactam or levofloxacin to the hydrophobin-coated stents was also tested. The coating of biliary plastic stents with hydrophobin or with hydrophobin and heparin was found to be a promising method to delay the clogging process.

If the clinical setting requires stenting over a longer period, endoscopic stent exchange is often necessary, but the development of hydrophobin-coated biliary stents with longer patency rates was found to be of particular use. In an in vitro model, different coatings of commercially available biliary plastic stents with a hydrophobin were tested. A reduction of the clogging process was found. Moreover, antibiotics and heparin were coupled to this protein hydrophobin to further improve the performance of the plastic stents. The concept resulted from the findings of scanning electron microsopic study which indicated that tiny threads seem to play a crucial role in the clogging process.

### EXAMPLE 1

For the following study, biliary plastic stents were used having with a diameter of 10 French and a length of 8 cm between flaps (polyethylene, type Cotton-Leung®, Fa. Cook® Medical).

Native plastic stents and hydrophbin-coated stents were placed in human bile for a period of 28 days. Afterwards the stents were examined by scanning electron microscopy.

Before the coating procedure was started, the stents were cut in pieces of 1 cm (suitable for final scanning electron microscopy). The surface modification and/or targeting of various actives were performed by a coating with a modified hydrophobin.

The generation and property of this protein “H*protein A” has been described in Wohlleben W, Subkowski T, Bollschweiler C et al. “Recombinantly produced hydrophobins from fungal analogues as highly surface-active performance proteins” in Eur Biophys J, 2009.

In the first experiments, 14 native stents were used. 11 stents were coated by general coating techniques with the hydrophobin protein (H*protein A, BASF SE) and 3 stents with hydrophobins (H*protein A in combination with ampicillin+sulbactam (Unacid®, Pfizer Pharma). Furthermore, the combinations H*protein A/levofloxacin (Tavanic®, Sanofi-Aventis) and H*protein A/heparin were tested.

After preparing a solution of 10 mg hydrophobin (H*protein A)/mL sterile distilled water, the protein was diluted to 1 mg/mL in the coating buffer: 50 mM Tris-HCl pH 8.0, 1 mM CaCl₂. The stents were incubated in 1 mL H*protein A solution overnight at 80° C., washed 3-times in distilled water and dried.

### EXAMPLE 2

### Detection of Hydrophobin on the Surface

The coating of H*protein A was detected by a specific antibody directed against the Hexa-His tag of the protein:
incubation of coated and uncoated stents in 1% blocking solution (Roche 1921673) in TTBS buffer (20 mM Tris-HCl pH 7.5, 150 mM NaCl, 0.05% Tween 20); 1 hr at room temperature (RT)
washing 3 times with TTBS buffer
incubation with anti-poly-His-POD antibody conjugate (Sigma A7058), diluted 1:2000 in TTBS buffer/0.01% BSA (Sigma A7888); 1 hr at RT
washing 2 times with TTBS buffer
washing with TBS buffer (20 mM Tris-HCl pH 7.5, 150 mM NaCl)
washing with 100 mM Na-acetate pH 4.9
addition of peroxidase substrate:

1 tablet TMB-substrate (Sigma T5525) in 100 μL DMSO
10 mL 0.1 M Na-acetate pH 4.9
14.7 μL 3% H₂O₂
5 min. incubation at RT
detection at 405 nm.

### EXAMPLE 3

### Coating of Plastic Stents with Hydrophobin and Heparin

The heparin was coupled to the H*protein A coating of the stents. A mixture of heparin (heparin sodium salt, Sigma H4784) and the coupling reagent 1-Ethyl-3-(3-dimethylaminopropyl)carbodiimid (EDC) (Sigma 7750) 1 mg/mL in 100 mM MES buffer (Sigma M2933) pH 4.5 was freshly prepared. The final concentration of heparin was 50 U/mL, 500 U/mL and 5000 U/mL. After incubation of the stents at RT for 2 hrs with 1 mL of the coupling solution in a rotator the reaction was stopped by 3 washing steps with sterile distilled water.

Via the free carboxyl group, the heparin was coupled to amino groups of H*protein A: EDC used as a carboxyl activating agent for the coupling of primary amines to yield amide bonds.

### EXAMPLE 4

### Stents Coated with H*Protein A and Ampicillin+Sulbactam (Unacid®, Pfizer Pharma

Unacid® was dissolved in sterile distilled water to a total concentration (ampicillin+sulbactam) of 100 mg/mL. The reaction with H*protein A on the surface of the stents was performed in distilled water by the addition of Unacid® to a final total concentration of 100 μg/mL and of glutardialdehyde (Aldrich 34,085-5) in a final concentration of 0.05%. After overnight incubation at RT, the stents were washed 3 times with sterile distilled water. Ampicillin as one of the 2 active compounds in Unacid® could be coupled under the reaction conditions: the primary amino group of ampicillin and accessible amino groups of the H*protein A are cross linked by glutardialdehyde.

### EXAMPLE 5

### Stents Coated with H*Protein A and Levofloxacin (Tavanic®, Sanofi-Aventis)

Tavanic® was diluted in 100 mM MES buffer pH 4.5 to a final concentration of 1.3 mg/mL and mixed with EDC (final concentration 1.6 mg/mL). The reaction with the H*protein A coated stents was performed at RT for 2.5 hrs. Finally the stents were washed 3 times with sterile distilled water.

The levofloxacin was coupled to the H*protein A by the generation of an amide: carboxyl group of levofloxacin cross linked to amino groups of H*protein A by EDC.

### EXAMPLE 6

### Incubation in Human Bile

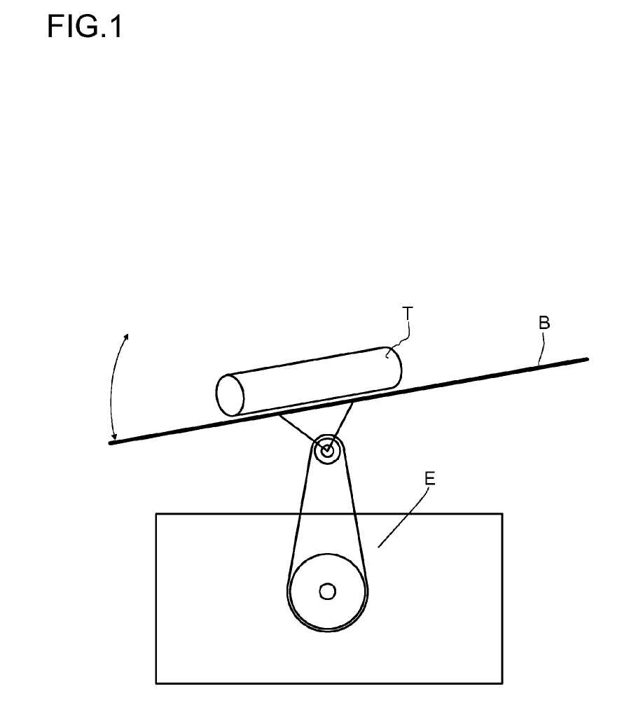

All coated stents were stored for 28 days in tubes filled with human bile (collected from percutaneous transhepatic drainage, the patients gave informed consent for the use of their bile). The tubes were turned with a rate of 2 rpm (rounds per minute) by using an apparatus as shown in FIG. 1.

In this FIG. 1, the abbreviation T is a tube with human bile storing stent, B is a board in slow pendulum movement, E is an electric motor. This treatment in the apparatus was done to simulate the bile flow. The incubation was performed at a constant temperature of 37° C.

### EXAMPLE 7

### Scanning Electron Microscopy

After 28 days of incubation all stents were washed with sterile distilled water, fixed and stored in 4% buffered glutaraldehyde. Following a graded series of alcohol for dehydration, all samples were dried using a critical point dryer, glued to stubs using adhesive foil and sputter coated with 15 nm gold-palladium. SEM-investigations were carried out using a Philips field-emission SEM with digital output.

First the findings of the uncoated stents were compared to the stents coated with H*protein A. Afterwards the stents coated with H*protein A were compared to the stents coated with H*protein A and the antibiotic drug compound or heparin.

The following results were found:
a) Uncoated stents

After the incubation period of 28 days scanning electron microscopy revealed similar amounts of adherent material on the inner and outer surface of the uncoated stents. The material was found to be more confluent on the inner stent surface, whereas it was more undulating on the outer surface. There was no difference with respect to the composition of the adhering material. The components were predominantly amorphous material with different amounts of bacteria and tiny structures in the proximity of bacteria as well as independent of their occurrence.
b) Stents coated with H*protein A

The modified hydrophobin used in this study consists of the hydrophobin DewA of Aspergillus nidulans, the N-terminal fusion protein yaad and the C-terminal Hexa-His tag. Due to specific antibodies against this tag sequence a “western blot like” detection of the protein on the surface could be performed. The enzyme POD (coupled to the antibody) could be detected by the color generation of a specific substrate.

### Detection of Optical Density at 405 nm

| uncoated stents (control) | coated stents |
|---|---|
| 0.10 | 0.59 |
| 0.14 | 0.68 |

The reduced extent of adhering material compared to the uncoated stents can be seen with low magnification by scanning electron microscopy.

Components demonstrated on uncoated stents are present also on the coated stents (amorphous material, bacteria and tiny structures in proximity and distant from the bacteria). A very thin layer (of hydrophobin) was detectable close to the stent surface.
c) Stents coated with H*protein A and ampicillin+sulbactam (Unacid®, Pfizer Pharma)

Even with higher magnification there was no indication of reduced adherent material in comparison to the stents coated with H*protein A alone. On the contrary, the amount of bacteria seemed rather to be pronounced on some stent segments.
d) Stents coated with H*protein A and Levofloxacin (Tavanic®, Sanofi-Aventis)

The scanning electron microscopy study of the stents revealed no difference to the stents coated with ampicillin+sulbactam. Therefore in this group there seemed to be no difference to the stents coated with H*protein A alone too.
e) Stents coated with H*protein A—heparin (concentrations: 50 U/ml, 500 U/ml and 5000 U/ml)

The stents coated with H*protein A and the lowest concentration of heparin had similar scanning electron microscopic findings as the stents coated with H*protein A alone. The stents coated with H*protein A and the highest concentration of heparin emerged as the stents with the least amount of adherent material. The stents coated with H*protein A and the intermediate concentration of heparin also showed good results.

The experiments show that the coating of plastic stents with the hydrophobin H*protein A led to a reduction of adherent material in comparison to uncoated plastic stents in the in vitro model. Coupling of hydrophobin with heparin in a high concentration reduced the adhesion process further.

As plastic stents have a hydrophobic surface, after coating with hydrophobins their surface becomes hydrophilic.

These properties are promising to optimize the surface of biliary plastic endoprostheses with respect to the inhibition of the clogging process. One of the particular useful hydrophobins is the H*Protein A, described in the Literature. Moreover it is possible to couple drugs to this H*Protein A.

The mechanism which led to the reduced adhesion in the in vitro model was studied. In recent scanning electron microscopy study a quite uniform clogging pattern of biliary and pancreatic stents after different stenting intervals was found. A crucial event in the clogging process is the adhesion of tiny threads to the inner stent surface and the stabilization of the clogging material by these tiny threads. The in vitro study of coatings of the stents led to a reduction of the amount of adherent material.

It was found that coupling of heparin to the hydrophobin in a high concentration reduced the adherent material. This can be confirmed in animal experiments.

To improve stent patency, different types of plastic have been investigated. Even a plastic stent with hydrophilic hydromer-coating and a seemingly smooth surface did not lead to a prolonged stent patency. The same was true for stents without sideholes (Tannenbaum Stent) as the sideholes had been suspected to accelerate stent clogging.

The stents treated with a composition according to the invention have smooth surfaces and lead to a prolonged stent patency.

1-15. (canceled)

16. A process for the coating of an implantable structure (I) comprising the step of treating the surface of the implantable structure (I) with a composition which comprises at least one hydrophobin derivative (H) of the general structural formula (I)
Xₙ—C¹—X₁₋₅₀—C²—X₀₋₅—C³—X₁₋₁₀₀—C⁴—X₁₋₁₀₀—C⁵—X₁₋₅₀—C⁶—X₁₋₅—C⁷—X₁₋₅₀—C⁸—Xₘ (I)
where X may be any of the 20 naturally occurring amino acids Phe, Leu, Ser, Tyr, Cys, Trp, Pro, His, Gln, Arg, Ile, Met, Thr, Asn, Lys, Val, Ala, Asp, Glu, Gly, where the X radicals may be the same or different in each case,
where the indices beside X are each the number of amino acids in the particular part-sequence X,
where C is cysteine, alanine, serine, glycine, methionine or threonine, where at least four of the residues designated with C are cysteine,
and where the indices n and m are each independently natural numbers between 0 and 500,
and wherein the implantable structure (I) is a stent.

17. The process for the coating of an implantable structure (I) according to claim 16, characterized in that the surface is treated with a composition which comprises at least one hydrophobin derivative (H) and at least one further component (F).

18. The process for the coating of an implantable structure (I) according to claim 16, wherein the composition comprises at least one hydrophobin derivative (H) and water, and potentially further components (F), whereby the amount of the hydrophobin derivative (H), based on the overall composition, is from 0.0001 to 20 percent by weight.

19. The process for the coating of an implantable structure (I) according to claim 16, wherein the implantable structure (I) is a stent which is treated with a composition which comprises at least one hydrophobin derivative (H), water, and potentially further components (F), whereby the amount of the hydrophobin derivative (H), based on the overall composition, is from 0.001 to 10 percent by weight.

20. The process for the coating of an implantable structure (I) according to claim 16, wherein a composition is applied to the surface of the implantable structure (I) which comprises the hydrophobin derivative (H), water, and at least one further pharmaceutically active compound (D).

21. The process for the coating of an implantable structure (I) according to claim 16, wherein a composition is applied to the surface of the implantable structure (I) which comprises the hydrophobin derivative (H), water, and a further pharmaceutically active compound (D) selected from the group consisting of heparin, antibiotics, and cytostatic compounds.

22. The process for the coating of an implantable structure (I) according to claim 16, wherein the hydrophobin derivative (H) used is a fusion hydrophobin or a derivative thereof.

23. The process for coating of an implantable structure (I) according to claim 16, wherein the composition comprising at least one hydrophobin derivative (H) is applied to the surface of the implantable structure (I) at a temperature from 4° C. to 95° C. for a time period of 0.01 hour to 48 hours.

24. A coated implantable structure (I) with a surface at least partially treated with a hydrophobin derivative (H) of the general structural formula (I)
Xₙ—C¹—X₁₋₅₀—C²—X₀₋₅—C³—X₁₋₁₀₀—C⁴—X₁₋₁₀₀—C⁵—X₁₋₅₀—C⁶—X₁₋₅—C⁷—X₁₋₅₀—C⁸—Xₘ (I)
where X may be any of the 20 naturally occurring amino acids Phe, Leu, Ser, Tyr, Cys, Trp, Pro, His, Gln, Arg, Ile, Met, Thr, Asn, Lys, Val, Ala, Asp, Glu, Gly, where the X radicals may be the same or different in each case,
where the indices beside X are each the number of amino acids in the particular part-sequence X,
where C is cysteine, alanine, serine, glycine, methionine or threonine, where at least four of the residues designated with C are cysteine,
and where the indices n and m are each independently natural numbers between 0 and 500,
wherein the implantable structure (I) is a stent.

25. A coated implantable structure (I) which is at least partially surface coated with a hydrophobin derivative (H) by the process according to claim 16, wherein the implantable structure (I) is a stent.

26. The coated implantable structure (I) according to claim 25, wherein the implantable structure (I) is a biliary stent.

27. A composition for the coating of an implantable structure (I), wherein the implantable structure (I) is a stent, and wherein the composition comprises 0.0001 to 20 percent by weight of hydrophobin (H) of the general structural formula (I)
Xₙ—C¹—X₁₋₅₀—C²—X₀₋₅—C³—X₁₋₁₀₀—C⁴—X₁₋₁₀₀—C⁵—X₁₋₅₀—C⁶—X₁₋₅—C⁷—X₁₋₅₀—C⁸—Xₘ (I)
where X may be any of the 20 naturally occurring amino acids Phe, Leu, Ser, Tyr, Cys, Trp, Pro, His, Gln, Arg, Ile, Met, Thr, Asn, Lys, Val, Ala, Asp, Glu, Gly, where the X radicals may be the same or different in each case,
where the indices beside X are each the number of amino acids in the particular part-sequence X,
where C is cysteine, alanine, serine, glycine, methionine or threonine, where at least four of the residues designated with C are cysteine,
and where the indices n and m are each independently natural numbers between 0 and 500,
and 99.999 to 80 percent by weight of further components (F), wherein the further components include water and at least one further pharmaceutically active compound (D) from the group comprising heparin, antibiotics and cytostatic compounds.

28. The composition according to claim 27, wherein the composition comprises at least 0.001 to 10 percent by weight of at least one hydrophobin derivative (H).
